(19) United States
(12) Patent Application Publication
(10) Pub. No.: US 2012/0238458 A1
(43) Pub. Date: Sep. 20, 2012

(54) Novel tumor marker determination

(75) Inventors: Dan CACSIRE CASTILLO-TONG, Wien (AT); Dietmar PILS, Wien (AT); Robert ZEILLINGER, Blumau-Neurisshof (AT); Eva OBERMAYR, Wien (AT)

(73) Assignee: Dr. Robert Zeillinger, Blumau-Neurisshof (AT)

(21) Appl. No.: 13/049,848

(22) Filed: Mar. 16, 2011

(51) Int. Cl. C40B 30/00; C12Q 1/28; G01N 33/573; C12Q 1/02; C12Q 1/68

(52) U.S. Cl. 506/7; 435/29; 435/6.12; 435/7.4; 435/6.14; 435/28

(57) ABSTRACT

A method of determining gynaecologic tumor disease in a subject by providing a sample of peripheral blood of the subject, measuring the PPIC expression of cells in the sample, and comparing this to a reference value, the PPIC overexpression being indicative of a gynecologic tumor disease and/or disease progression, including metastatic potential in a gynecologic cancer patient.

### FIELD OF THE INVENTION

The present invention relates to a method for determining gynaecologic tumor disease in a subject at risk of such disease.

A tumor marker, also called marker or biomarker, is a substance sometimes found in an increased amount in the blood, other body fluids, or tissues and which may mean that a certain type of cancer is in the body. There are many different tumor markers, each indicative of a particular disease process, and they are used in oncology as a diagnostic, prognostic, or predictive marker or used to monitor cancer therapy.

Often, tumor-specific markers are overexpressed in tumor tissue. Thus, the expression of tumor-specific genes in cancerous tissue is investigated to gain information about prognostic markers and molecular targets for diagnosis or chemical and/or immunological therapy.

Almost two million women in the whole world are diagnosed with breast cancer or other gynecologic malignancies, such as cervical, endometrial, and ovarian cancer each year. Although several advances have been made in early diagnosis during the past few decades, many patients still die of metastasis being the main cause for tumor-related death. In these patients, hematogenous spreading of malignant cells remained undetected at the time of initial diagnosis. This is of particular importance in the case of ovarian cancer with a high metastatic potential.

The use of markers such as estrogen receptor and HER2 for the selection of breast cancer treatment is well established. Recent interest in personalized therapy has led to the search for new markers of prognostic or predictive value.

Tumor cells circulating in the blood of cancer patients, also called circulating tumor cells (CTC) or disseminated tumor cells (DTC), have been described for a series of solid tumor diseases, such as colorectal, lung, kidney, squamous oesophageal, liver, prostate and pancreatic malignancies. Among gynecologic malignancies, most of the research has been done on CTC in breast cancer, whereas only a few data exist on CTC in ovarian, cervical and endometrial cancer. Christofanilli et al. (J Clin Oncol, 2005. 23(7): p. 1420-30) showed that the detection of CTC can predict patient outcome, and the presence of tumor cells in the peripheral blood was considered to be established as an additional staging parameter. For these reasons, many efforts have been made to develop reliable procedures for the sensitive and specific detection of CTC, either at the protein level, e.g. antibody-based cell staining, or at the mRNA level, e.g. reverse transcription PCR. While the first approach is the gold standard technique for the detection of tumor cells in the bone marrow of breast cancer patients, the latter is supposed to be more sensitive and amenable to high-throughput analysis.

The presence of CTC in a cancer patient undergoing therapy or after therapy would allow the definition of residual disease risk and thereby indicate the potential value of additional or alternative treatment. Minimal residual disease is determined by residual malignant cells; even when so few cancer cells are present that they cannot be found by routine means. Tests for minimal residual disease can detect some early tumors. Persistence of minimal residual disease after primary treatment may be an indication for extensive adjuvant treatment in order to prevent relapse of the disease. Patients, who need intensive and potentially more toxic therapy, can be distinguished from those who do not.

Klein C A (Adv. Cancer Res. 2003; 89:35-67) describe that DTC do not necessarily have the same molecular characteristics as the primary tumors. In contrast, cancer cell evolution obviously explores a multitude of variant cells from which systemic cancer can develop independently. Thus, markers derived from studying the expression profile of tumor tissue would not necessarily be determined in blood samples.

The object of the present invention was to find a new biomarker to evaluate the risk of a gynaecologic tumor in a subject.

The object is achieved by the provision of the embodiments of the present invention.

### SUMMARY OF THE INVENTION

The present invention refers to a method of determining gynaecologic tumor disease in a subject, which comprises

providing a sample of peripheral blood of the subject,

measuring the PPIC expression of cells in said sample, and

comparing to a reference value,

the PPIC overexpression being indicative of a gynecologic tumor disease and/or disease progression.

Specifically the sample is obtained from a blood fraction containing mononuclear cells, such as a PBMC fraction. Such a PBMC fraction may be obtained through fractionating blood for the enrichment of blood cells and—amongst others—CTC or epithelial cells, if present.

In a preferred embodiment the method according to the invention further comprises additionally determining CDH3 expression of said CTC, wherein the CDH3 expression is indicative of an improved prognosis. This is of particular importance in determining ovarian tumor disease.

According to a specific aspect at least one further marker selected from the group consisting of GPX8, TUSC3, AGR2, COL3A1, LAMB1, MAM (also called SCGB2A2), TFF1, BAIAP2L1, ESRP2 and EpCAM is determined, e.g. in the same sample, optionally employing a set of reagents for the simultaneous or parallel determination of a respective marker panel. If a reduced panel is preferred, the further markers of MAM and EpCAM are less preferred than others or even excluded, because they appear less specific.

Specifically the nucleic acid and/or protein expression is determined, either qualitatively or quantitatively. In a preferred embodiment the PPIC expression is quantitatively determined. In a quantitative determination the significant overexpression of the PPIC and optionally further biomarkers is eventually measured. In a preferred embodiment the method according to the invention employs an internal standard that is measured either side-by-side with the sample determination or used to calibrate the determination system. This is particularly preferred for the quantitative determination method.

The expression is optionally employing amplification methods, among them signal or nucleic acid amplification methods, RT-qPCR, microarrays, immunoassays, such as ELISA, EIA, RIA, western blot, protein arrays, immunocytochemistry or immunohistochemistry methods, as appropriate.

In a preferred method according to the invention, the determination method is highly sensitive, e.g. with a detection limit of a biomarker in a sample containing less than 30 tumor cells/ml blood, preferably less than 15 tumor cells/ml, preferably less than 7 tumor cells/ml or less than one tumor cell/ml, even to a detection limit of one tumor cell per 25 ml or less. In a preferred method the determination method is employed in a sample containing at least one tumor cell per 25 ml blood, specifically one tumor cell/ml whole blood.

In a further preferred method, the reference value is determined in a sample of a healthy subject, e.g. from a potentially epithelial cells containing blood fraction of the healthy subject, specifically in a sample containing mononuclear cells, or otherwise derived from a healthy or control subject.

The preferred method according to the invention provides for the comparison of the results of a marker gene expression (determined qualitatively, semi-quantitatively or quantitatively) with a reference value or level. A preferred embodiment comprises a comparative gene expression analysis or the comparison of a gene expression pattern. In a further preferred method, the amount of overexpression differs significantly from the reference value, e.g. at least 1.5 times of the reference value.

According to one aspect of the invention, the subject is at risk of ovarian, breast, endometrial or cervical tumor disease, including disease development or progression of such a disease. Samples are preferably taken from patients who are actually suffering from cancer, in particular who have been diagnosed with cancer.

Yet, according to a specific aspect, the method according to the invention is used for the diagnosis of gynecologic cancer, e.g. for diagnosing early stage cancer. Specifically the subject is suffering from early stage cancer. Thereby a diagnosis, prognosis or prediction is first time possible in such subjects based on the method according to the invention.

According to another aspect of the invention, the subject said subject is undergoing or has received chemotherapy, and the risk of disease progression is determined.

Specifically the subject is suffering from minimal residual disease.

According to another aspect of the invention, the PPIC overexpression is indicative of an increased metastatic potential associated with a shortened survival time, either overall or progression-free survival time.

Thus, the method of the invention is preferably used for the diagnosis of the metastatic potential in a gynecologic cancer patient at risk of disease progression.

According to the invention a set of reagents is preferably used for detecting circulating tumor cells in a subject, which allows the determination of the PPIC expression marker, and optionally further markers, which includes protein binders and/or nucleic acids. The set of reagents according to the invention preferably comprises ligands, such as antibodies, antibody fragments, or hybridisation probes, which are optionally labelled. The preferred set of reagents also includes an internal standard for the eventual quantitative determination.

### DETAILED DESCRIPTION

Peptidyl-prolyl-isomerase C (PPIC)

The protein encoded by this gene located on chromosome 5q23.3 is a member of the peptidyl-prolyl cis-trans isomerase (PPlase) family and is also called cyclophilin C (CypC). Cyps regulate protein folding through PPlase enzymatic and chaperone activities in specific locales of the cells to ensure correct conformation and to counterbalance conformational variations under diverse stress conditions. In addition to PPlase and chaperone activities, each isoform of Cyps has other specific intracellular and extracellular roles. Although roles of Cyps have recently been explored in more details, many physiological and pathological aspects of Cyps' biology still remain unclear [3].

CypC is another Cyp family member that is primarily located in ER, but its role remains to be determined. CypC can form a complex with the COOH-terminal fragment of osteopontin. This complex binds to CD147 to activate Akt1/2 and MMP-2 in 4T07 murine breast cancer cells. This CypC-osteopontin complex regulates in vitro migration and invasion properties of 4T1 and 4T07 breast cancer cells [4].

The PPIC gene or expression product was heretofore not associated with tumor disease or cancer. It was thus surprising that CTC would overexpress PPIC as a diagnostic marker indicating the presence of a gynecologic tumor.

CDH3

The CDH3 gene encoding the cadherin 3, type 1 protein, which is also called P-cadherin is located on the 16q22.1 cytoband. This gene is a classical cadherin from the cadherin superfamily. The encoded protein is a calcium-dependent cell-cell adhesion glycoprotein comprised of five extracellular cadherin repeats, a transmembrane region and a highly conserved cytoplasmic tail. This gene is located in a six-cadherin cluster in a region on the long arm of chromosome 16 that is involved in loss of heterozygosity events in breast and prostate cancer. In addition, aberrant expression of this protein is observed in cervical adenocarcinomas. Mutations in this gene have been associated with congential hypotrichosis with juvenile macular dystrophy.

Cadherins are calcium dependent cell adhesion proteins. They preferentially interact with themselves in a homophilic manner in connecting cells; cadherins may thus contribute to the sorting of heterogeneous cell types. P-cadherin-mediated adherens junctions and the associated signaling pathway play diverse roles in the regulation of tumor cell proliferation, invasiveness and metastatic potential. Upregulation of P-cadherin was frequently observed in various malignancies, including breast, colon, lung and pancreatic tumors, and P-cadherin increase correlated with poor survival of breast cancer patients (Zhang C C, Clin Cancer Res. 2010 16(21):5177-88).

CDH3 was surprisingly found to be an additional prognostic marker according to the invention. CDH3 overexpression of circulating cells actually indicated a decreased risk of developing gynecologic cancer, in particular ovarian cancer, or an improved prognosis.

The term “biomarker” or “biomarker according to the invention” as used herein shall refer to PPIC and optionally CDH3, possibly combined with further biomarkers, such as selected from the panel GPX8, TUSC3, AGR2, COL3A1, LAMB1, TFF1, BAIAP2L1 and ESRP2, optionally further including MAM and/or EpCAM.

The term “PPIC overexpression” as used herein shall refer to samples or cells expressing a higher amount of PPIC, specifically a significantly higher amount, as compared to a reference value, which may be zero or higher, e.g. higher than a threshold or cut-off value, or higher than a reference value derived from a comparable sample. Overexpression may as well be determined by comparison to standards, including internal or external standards.

The term “significantly higher” or “significant” with respect to the overexpression of a biomarker as used herein shall refer to at least a two-fold higher amount of the standard deviation, preferably at least a three-fold difference. With respect to a specific reference value, such as a threshold, a significant increased amount is understood to refer to an at least 1.5 fold higher amount, preferably at least 2 or 3 fold difference.

The term “gynaecologic tumor” as used herein shall refer to a tumor of female sexual organs, including ovarian, breast, endometrial or cervical tumor, bit also encompass male breast tumors. Such tumors may be benign or malignant, including cancer.

The term “PBMC fraction” as used herein shall refer to a fraction of peripheral blood including peripheral blood mononuclear cells, either enriched or not, e.g. enriched in white monocytes by density gradient centrifugations. The term shall also include PBMCs preparations isolated from whole blood, e.g. purified PBMC preparations that may be enriched or depleted from CTC that are eventually present in the blood samples.

Thus, the present invention provides for the determination of the PPIC marker alone, or with one or more members of a panel of biomarkers that can be used in a method for determining the disease condition, including detection, diagnosis, in particular early stage diagnosis, prognosis, therapy response prediction, or monitoring solid tumor disease, monitoring therapy, for determining the disease stage, including metastatic disease, and disease status.

In specific aspects of the method of the invention, the method is non-invasive for gynecologic tumor diagnosis, which in turn allow for diagnosis of a variety of conditions or diseases associated with solid tumor disease. In particular, the invention provides a non-invasive non-surgical method for determining the disease condition.

Preferred marker combinations can be derived from the examples below, which are highly specific, e.g. identifying true positive patients, such as a specificity of 50%, 60%, 70% or even more preferred at least 80%. Any marker combination of at least PPIC and optionally one or more markers associated with cancer, which brings about a ratio of positive patients as described above, is considered a preferred combination to determine the risk of gynecologic tumor development.

In a specifically preferred method according to the invention, at least one further marker selected from the group of GPX8, TUSC3, AGR2, COL3A1, LAMB1, MAM (also known as SCGB2A2), TFF1, BAIAP2L1, ESRP2 and EpCAM is determined.

GPX8

The GPX8 gene is located on chromosome 5q11.2 and encodes for the putative glutathione peroxidase 8. This protein belongs to the glutathione peroxidase family and is integrated into the cytoplasmic membrane. The family members 1 to 7 play diverse roles in tumor cells, whereas little is known about the role of GPX8 (Toppo S et al., Antioxid Redox Signal. 2008 September; 10(9):1 501-14. and Brigelius-Flohé Ret al., Biochim Biophys Acta. 2009 November; 1790(11):1555-68).

TUSC3

This gene is a candidate tumor suppressor gene. It is located on chromosomal band 8p22, where losses of heterozygosity are frequently observed in epithelial tumors including ovarian carcinoma. The gene is expressed in most nonlymphoid human tissues including prostate, lung, liver, and colon. Expression was also detected in many epithelial tumor cell lines. Two transcript variants encoding distinct isoforms have been identified for this gene. Methylation of promotor-associated CpG island leading to the inactivation of tumor suppressor genes was observed in glioblastoma multiforme (Li Q et al, Oncogene 1998 16(24):3197-202).

AGR2

Human AGR2 is a homolog of the secreted Xenopus laevis protein (XAG-2). In Xenopus, XAG-2 is primarily involved in the induction and differentiation of the cement gland, as well as in the patterning of anterior neural tissues [5]. The human AGR2 gene is located on the chromosomal band 7p21.3. AGR2 gene expression was observed in human tissues rich in epithelial cells. Overexpression occurs in the majority of tumors as compared to matched adjacent benign tissues. Smirnov et al. identified AGR2 as a potential marker for detection of circulating tumor cells in the blood of patients with metastatic cancers [6].

COL3A1

This gene encodes the pro-alphal chains of type III collagen, a fibrillar collagen that is found in extensible connective tissues such as skin, lung, uterus, intestine and the vascular system, frequently in association with type I collagen. Mutations in this gene, which is located on 2q31, are associated with Ehlers-Danlos syndrome types IV, and with aortic and arterial aneurysms. Two transcripts, resulting from the use of alternate polyadenylation signals, have been identified for this gene. Differences in gene expression were observed between advanced and local ovarian carcinoma (up-regulation in advanced stages). The differential gene expression may be related to the carcinogenesis and progression of the malignant growth (Tapper J et al., Cancer Genet Cytogenet, 2001 128(1):1-6).

LAMB1

Laminins, a family of extracellular matrix glycoproteins, are the major noncollagenous constituent of basement membranes. They have been implicated in a wide variety of biological processes including cell adhesion, differentiation, migration, signalling, neurite outgrowth and metastasis. Laminins are composed of three non-identical chains: laminin alpha, beta and gamma (formerly A, B1, and B2, respectively) and they form a cruciform structure consisting of three short arms, each formed by a different chain, and a long arm composed of all three chains. Each laminin chain is a multidomain protein encoded by a distinct gene. Several isoforms of each chain have been described. Different alpha, beta and gamma chain isomers combine to give rise to different heterotrimeric laminin isoforms, which are designated by Arabic numerals in the order of their discovery, i.e. alpha1beta1gamma1 heterotrimer, is laminin 1. The biological functions of the different chains and trimer molecules are largely unknown, but some of the chains have been shown to differ with respect to their tissue distribution, presumably reflecting diverse functions in vivo. This gene encodes the beta chain isoform laminin, beta 1. The beta 1 chain has seven structurally distinct domains, which it shares with other beta chain isomers. The C-terminal helical region containing domains I and II are separated by domain alpha, domains III and V contain several EGF-like repeats, and domains IV and VI have a globular conformation. Laminin, beta 1 is expressed in most tissues that produce basement membranes, and is one of the three chains constituting laminin 1, the first laminin isolated from Engelbreth-Holm-Swarm (EHS) tumor. A sequence in the beta 1 chain that is involved in cell attachment, chemotaxis, and binding to the laminin receptor was identified and shown to have the capacity to inhibit metastasis.

MAM (SCGB2A2)

SCGB2A2 [7], widely known as mammaglobin, is a member of the secretoglobin subfamily [8], a group of small, secretory, rarely glycosylated, dimeric proteins mainly expressed in mucosal tissues, and that could be involved in signalling, the immune response, chemotaxis [9] and possibly, as a carrier for steroid hormones in humans.

SCGB2A2 expression has rarely been found in healthy individuals. Thus, it has become the most widely studied marker in DTC detection after CK19, at least in breast cancer patients. At the same sensitivity as CK19 [10], patients are identified with 100% specificity. Nevertheless, mammaglobin expression is highly variable in female cancers and is detected in the blood of only 10 to 30% breast cancer patients. Unfortunately, the most aggressive, steroid receptor-negative, high-grade breast tumors and their corresponding CTC are likely to escape detection using SCGB2A2 as marker.

Zafrakas et al [11] found SCGB2A2 abundantly expressed in tumors of the female genital tract, i.e. endometrial, ovarian and cervical cancer. This observation might extend the diagnostic potential of SCGB2A2 to the detection of CTC from gynecologic malignancies.

TFF1

Members of the trefoil family are characterized by having at least one copy of the trefoil motif, a 40-amino acid domain that contains three conserved disulfides. They are stable secretory proteins expressed in gastrointestinal mucosa. Their functions are not defined, but they may protect the mucosa from insults, stabilize the mucus layer, and affect healing of the epithelium. This gene, which is expressed in the gastric mucosa, has also been studied because of its expression in human tumors. This gene and two other related trefoil family member genes are found in a cluster on chromosome 21. TFF1 expression is correlated with steroid receptor status and elevated transcript levels have been observed in various neoplastic tissues, including breast cancer [12].

BAIAP2L1

This gene is located on 7q22.1 and encodes a member of the IMD (IRSp53/MIM homology domain) family. Members of this family can be subdivided in two groups, the IRSp53-like and MIM-like, based on the presence or absence of the SH3 (Src homology 3) domain. The protein encoded by this gene contains a conserved IMD, also known as F-actin bundling domain, at the N-terminus, and a canonical SH3 domain near the C-terminus, so it belongs to the IRSp53-like group. This protein is the substrate for insulin receptor tyrosine kinase and binds to the small GTPase Rac. It is involved in signal transduction pathways that link deformation of the plasma membrane and remodelling of the actin cytoskeleton. It also promotes actin assembly and membrane protrusions when overexpressed in mammalian cells, and is essential to the formation of a potent actin assembly complex during EHEC (Enterohemorrhagic Escherichia coli) pedestal formation.

ESRP2

ESPR2 is an epithelial cell-type-specific splicing regulator. Epithelial- and mesenchymal-specific isoforms that are regulated by the ESRPs are likely to participate in epithelial-mesenchymal crosstalk during early vertebrate development and to have important roles in epithelial to mesenchymal transitions during development as well as in disease processes such as cancer metastasis and tissue fibrosis [13].

EPCAM (Epithelial Cell Adhesion Molecule)

This gene encodes a carcinoma-associated antigen and is a member of a family that includes at least two type I membrane proteins. This antigen is expressed on most normal epithelial cells and gastrointestinal carcinomas and functions as a homotypic calcium-independent cell adhesion molecule. Because of its ubiquitous expression on the surface of epithelial cells, EPCAM can be considered as a pan-carcinoma tumor marker. The antigen is used as a target for immunotherapy treatment of human carcinomas.

EPCAM has been frequently used as target for positive immunomagnetic separation to enrich tumor cells for RT-PCR analysis. Monoclonal antibodies against this antigen have been extensively developed for diagnostic (CellSearch), but also therapeutic, approaches. Although highly sensitive for epithelial malignancies, including breast cancer, however, its use for CTC detection is hampered by the fact that it is expressed in low amounts in peripheral blood cells. Furthermore, it has been shown that the normal-like breast cancer cells characterized by aggressive behaviour and worse treatment options are not recognized by the CellSearch test (Veridex, LLC), which is the only diagnostic test for circulating tumor cells currently approved by the US Food and Drug Administration and which utilizes an anti-EpCAM antibody.

According to a specific aspect there is preferably provided a set of reagents to determine the biomarker according to the invention.

Preferably, means for determining a biomarker or the expression pattern or expression signature according to the invention are employed to provide a multi-marker panel, comprising at least two biomarkers according to the invention, comprising PPIC and optionally CDH3 and further biomarkers, which may be particularly used for determination of ovarian tumor disease.

This panel according to the invention preferably further comprises one or more markers selected from the group consisting of GPX8, TUSC3, AGR2, COL3A1, LAMB1, MAM (SCGB2A2), TFF1, BAIAP2L1, ESRP2 and EpCAM.

In a specific embodiment, the invention contemplates marker panels containing or consisting essentially of at least two, three, four, five, six, or more, wherein at least one of the biomarkers is PPIC. The inventive panel preferably includes only those biomarkers that are associated with gynecologic tumor disease, preferably only those that would differentiate between patients having detectable CTCs associated with malignancy or metastasis and healthy subjects, which eventually have non-tumor derived cells in a body fluid sample. The multimarker panel preferably comprises the biomarker polypeptide or gene sets.

The term “subject” as used herein shall refer to any mammal, in particular a human but also selected from animals, such as those used for tumor models and other animal studies. Preferably, the subject shall be human beings, in particular female, who are patients at risk of a gynaecologic tumor, including benign or malignant lesions The term “patient” herein always includes healthy subjects.

A subject “at risk of” tumor disease is herein understood as a subject that has an already diagnosed or undiagnosed tumor, including those already suffering from such a disease at various stages, including the early stage and advanced disease state, particularly associated with malignant tumors, or else subjects that develop a progressive disease In accordance therewith the determination of the risk of gynaecologic tumor is herein specifically referring to diagnosis, prognosis and/or prediction of therapy response.

The risk determination to diagnose a gynecologic tumor is particularly important in a subject, where a gynecologic malignancy has not yet been diagnosed. This risk determination therefore includes early stage diagnosis. Preferably, those patients are tested for the biomarker according to the invention, before a solid tumor is detected, or before malignancy has proven by biopsy, where no cancerous disease is diagnosed.

Healthy subjects are usually not tested for any tumor disease biomarkers in the absence of any detectable tumor. However, there are high risk subjects, who have an increased risk of developing a gynecologic tumor disease because of a genetic predisposition or other risk factors, including age, life style, family predisposition or history. Antecedent diseases, such as cancer, or benign tumors or certain medical treatment would also increase the risk of developing solid tumors and associated disease conditions. Several risk factors for solid tumors that classify a high cancer risk, e.g. breast cancer, have been identified so far, among them BRCA1-, BRCA2-, p53-gene mutations, hormonal therapies, etc.

The early detection of solid tumor disease is essential in the patient population that is already classified as high-risk patients. It is thus preferred to test a patient population according to the invention, which is already classified as risk patients.

In particular, the inventive method allows the early stage determination of the solid tumor disease or respective risk stages, e.g. to distinguish between low, medium and high risk patients.

In advanced cancer disease but also in minimal residual disease, the risk of relapse can be high, which is usually associated with poor prognosis. Thus, the risk determination according to the invention particularly refers to the prognosis of a subject to develop cancer relapse and/or the prognosis of a cancer patient, and in particular to the determination of the metastatic potential.

The method according to the invention is specifically provided for determining susceptibility to gynecologic cancer or the risk of gynecologic tumor disease, in a subject comprising:

(a) obtaining a blood sample from a subject,

(b) detecting or identifying in the sample PPIC and eventual further biomarkers of the panel of the invention, and

(c) comparing the detected amount with a standard amount or an amount detected for a reference.

The term “detect” or “detecting” includes assaying, imaging or otherwise establishing the presence or absence of the target biomarker. The level of biomarkers or amount of biomarkers is herein understood to refer to the respective polypeptides or nucleotide sequence, including variants such as splice variants, subunits thereof, or reagent bound targets.

The target biomarker is preferably determined by testing for the respective polypeptides and/or polynucleotides indicative of marker expression. The expressed marker is detectable e.g. as polynucleotide, like mRNA, or expressed polypeptide or protein. The comparison with the reference value should be of the same sample type. Thus, the reagents preferably comprise ligands specifically binding to the biomarker polypeptide or gene or genetic marker, e.g. comprising a plurality of respective polypeptides, genes or polynucleotides. Ligands are herein understood as marker specific moieties.

Marker specific moieties are substances which can bind to or detect at least one of the markers for a detection method described above and are in particular marker nucleotide sequence detecting tools or marker protein specific antibodies, including antibody fragments, such as Fab, F(ab), F(ab)′, Fv, scFv, or single chain antibodies. The marker specific moieties can also be selected from marker nucleotide sequence specific oligonucleotides, which specifically bind to a portion of the marker sequences, e.g. mRNA or cDNA, or are complementary to such a portion in the sense or complementary anti-sense, like cDNA complementary strand, orientation.

The preferred ligands may be attached to solid surfaces, including beads, to catch and separate the marker or CTC in the sample, and/or to labels. Biological assays require methods for detection, and one of the most common methods for quantitation of results is to conjugate a detectable label to a protein or nucleic acid that has affinity for one of the components in the biological system being studied. Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.).

In particular aspects of the invention, the methods described herein utilize PPIC and optionally one or more markers of a multimarker panel placed on a microarray so that the expression status of each of the markers is assessed simultaneously. In an embodiment, the invention provides a microarray as a prognostic tool comprising a defined set of marker genes, whose expression is significantly altered in gynecologic cancer and which may be determined by hybridization or by amplification of polynucleotides.

In preferred embodiments, the mRNA concentration of the marker(s) is determined. To this extent, mRNA of the sample can be isolated, if necessary, after adequate sample preparation steps, e.g. tumor cell enrichment and/or lysis, and hybridized with marker specific probes, in particular on a microarray platform with or without amplification, or primers for PCR-based detection methods, e.g. PCR extension labelling with probes specific for a portion of the marker mRNA.

The invention preferably contemplates a gene expression profile comprising a multimarker panel that is associated with gynecologic cancer, in particular ovarian cancer. This profile provides a highly sensitive and specific test with both high positive and negative predictive values permitting diagnosis and prediction of the patient's risk or the risk of developing metastatic disease.

For example, the invention provides a method for determining the risk of gynecologic tumor disease in a subject comprising

(a) contacting a blood sample obtained from said subject with one or more oligonucleotides that hybridize with one or more markers, which are PPIC and optionally one or more of the markers of the multimarker panel described above, and

(b) detecting in the sample a level of polynucleotides that hybridize to the one or more markers relative to a reference level or predetermined cut-off value, and therefrom determining the risk of developing a gynecologic tumor in the subject.

Within certain preferred embodiments, the amount of mRNA is detected via polymerase chain reaction using, for example, oligonucleotide primers that hybridize to a marker gene, or complements of such polynucleotides. When using mRNA detection, the method may be carried out by combining isolated mRNA with reagents to convert to cDNA according to standard methods and analyzing the products to detect the marker presence in the sample.

In further embodiments the amount of a marker or any combination thereof is determined by the polypeptide or protein concentration of the marker(s), e.g. with marker specific ligands, such as antibodies or specific binding partners. E.g., the binding event can be detected by competitive or non-competitive methods, including the use of labeled ligand or marker specific moieties, e.g. antibodies, or labeled competitive moieties, including a labeled marker standard, which compete with marker proteins for the binding event. If the marker specific ligand is capable of forming a complex with the marker, the complex formation indicates expression of the markers in the sample.

In particular, the invention relates to a method for diagnosing and monitoring gynecologic tumor disease in a patient by quantitating a marker in a blood sample from the patient comprising

(a) reacting the sample with one or more binding agents specific for PPIC and optionally one or more markers of the multimarker panel according to the invention, e.g. an antibody or antibody fragment that is directly or indirectly labelled with a detectable substance, and

(b) detecting the detectable substance.

The preferred method employs an immunoassay. In general, immunoassays involve contacting a sample potentially containing a biomarker of interest with at least one immunoligand that specifically binds to the marker. A signal is then generated indicative of the presence or amount of complexes formed by the binding of polypeptides in the sample to the immunoligand. The signal is then related to the presence or amount of the marker in the sample. Immunoassays and respective tools for determining PPIC and the other markers are well-known in the art.

The invention also relates to kits for carrying out the methods of the invention, specifically those including a reference or a standard.

The invention further contemplates the methods, compositions, and kits described herein using additional markers associated with gynaecologic or other epithelial cancer. The methods described herein may be modified by including reagents to detect the additional markers, or polynucleotides for the markers.

Preferably, the differential marker expression is determined by comparing the expression to the control of healthy subjects or patients suffering from a benign tumor.

Reference values for the biomarker are preferably obtained from a control group of subjects with normal expression of said biomarker, or a biomarker expression, that is associated with the disease condition, such as disease stages, which represents the appropriate reference value. In a particular aspect, the control comprises material derived from a pool of samples from normal subjects. The normal level of a biomarker may be determined in samples of the same type obtained from control subjects.

The reference values are typically calculated from standard deviations of the mean average marker expression in healthy subjects. If more than one marker is detected, the comparison is made to each single reference value for each marker in the reference itself. The risk of the presence of a gynecologic tumor may be indicated if the amount of the biomarker or the combination of markers is significantly increased as compared to a standard, e.g. derived from a non-tumor cell, or reference value of subjects not suffering from gynecologic tumor, preferably being subjects from a control group or healthy subjects. The risk determination typically correlates with the level of overexpression. If at least two biomarkers of the panel according to the invention are increased, the risk is considered to be even more increased. With regard to the PPIC and the other markers, overexpression indicates an increased risk of the presence of a gynecologic tumor disease. In addition, an increase of CDH3 overexpression may indicate a prolonged survival.

The marker level can also be compared to a threshold, e.g. a cut-off concentration and the gynecologic likelihood for the presence for a tumor disease is determined from such comparison; wherein the PPIC concentration above the reference value is predictive of tumor presence in the patient.

In a preferred quantitative determination method, the expression of biomarkers is normalized to the median expression of one or more reference genes, used as internal control.

Thus, the preferred method according to the invention comprises the step of comparing the PPIC marker level with a predetermined standard or cut-off value, which is preferably at least 25% higher than the standard, more preferred at least 40% or 50% higher, but can also be at least 100% higher.

The higher the fold increase of PPIC, the higher is the patient's risk of having such gynecologic tumor disease. An elevated PPIC value alone or in combination with the other markers of the panel according to the invention indicates, for example, special treatment of the patient, using appropriate medication or further diagnostic techniques, such as imaging and surgical interventions. The method of the invention can thus be used to evaluate a patient before, during, and after medical treatment.

Types of cancer treatment that are used as adjuvant therapy include chemotherapy, hormone therapy, radiation therapy, immunotherapy or targeted therapy. Following first line chemotherapy, for instance, the cancer patient can be determined for the metastatic potential to decide about a second line adjuvant treatment.

According to a specific embodiment, the numbers of CTC in blood is determined for diagnostic or prognostic purposes. The CTC may be enriched in a body fluid, in particular blood, and the expression profile of the cells is determined. When a ligand specifically binding to the biomarker is used as capturing agent, the CTCs are preferably enriched and optionally isolated before testing the marker expression, and the test results are evaluated, e.g. according to the CTC epithelial cell functions or properties.

The enrichment of CTC is particularly preferred for genome analysis or molecular analysis employing nucleic acids as probes to hybridize with the specific biomarkers. For instance, RT-PCR or RT-qPCR is preferably employed. Upon enrichment of CTC, the RNA can be analyzed. For example, disseminated, circulating tumor cells from peripheral blood are enriched using a cell separation procedure to produce a fraction prior to sample analysis. A standardized system for tumor cell enrichment is e.g. provided as OncoQuick® (Greiner Bio-One, Frickenhausen, Germany). Thereby a fraction of mononuclear cells is obtained, optionally co-enriched with tumor cells and subsequent nucleic acid or immunocytochemical evaluation is possible with high sensitivity.

The invention also contemplates a method of assessing the potential of a test compound to contribute to gynecologic cancer therapy. For instance, an ex vivo method according to the invention may comprise the following steps:

(a) maintaining separate aliquots of a blood sample from a patient in the presence and absence of the test compound, and

(b) comparing the levels of PPIC and optionally of one or more further biomarkers, e.g. a multimarker panel in each of the aliquots.

This method may also be particularly useful as an in vivo method in monitoring the marker level in non-human animal models, or during clinical trials. A significant difference between the levels of a marker in an aliquot maintained in the presence of or exposed to the test compound relative to the aliquot maintained in the absence of the test compound, indicates that the test compound potentially contributes to gynecologic cancer therapy.

The present invention is further illustrated by the following example without being limited thereto.

### EXAMPLE

### Prognosis of Ovarian Cancer Patients

In the first step, a whole genome expression analysis of ovarian cancer tissues (35 patients) and of peripheral blood mononuclear cells (20 healthy female donors, 35 patients) using microarrays was performed. From 40 gene markers with low/absent gene expression PBMCs and high/present gene expression in ovarian cancer tissues 25 markers were selected for further analysis. Additionally, 15 markers were selected due to results of a previous study aiming at defining molecular markers for the detection of CTCs in breast cancer and gynecologic cancer (ovarian, endometrial and cervical cancer).

As a reference, the gene expression of the 40 differentially expressed genes was validated in PBMCs obtained from 20 patients with benign ovarian diseases with RT-qPCR. As a result, 11 gene markers remained in the study due to absent/low gene expression in benign PBMCs.

Then the gene expression of these 11 markers was analysed in the blood of 248 ovarian cancer patients and 39 healthy females. EpCAM (Epithelial cell adhesion molecule) was analysed as additional gene marker to compare results obtained from RT-qPCR and from immunocytochemistry. 230 blood samples were taken before initial surgery of the primary tumor, and 115 blood samples six months after completion of the first line adjuvant chemotherapy. From 97 patients blood samples taken at both timepoints were available.

Gene expression was normalized to the median expression of three reference genes (B2M, ACTB, and TBP). Due to background gene expression in the healthy PBMC samples, a threshold TX was introduced to separate healthy and diseased individuals and to identify CTC-positive patients as proposed by Mikhitarian et al. (BMC Cancer 2008, 8:55). A patient was defined as CTC-positive if an at least one out of 11 gene marker over-expressed.

As a result, 56/230 patients were positive before initial surgery, but only 21/115 six months after chemotherapy (Table 1). Detailed results for the respective gene markers are shown in Table 2. The presence of CTC as indicated by RT-qPCR was correlated to clinical parameters of the patients (age, response to adjuvant chemotherapy, residual tumor after surgery, FIGO, peritonealcarcinomatosis, ascites, and recurrence during follow-up). Significantly more patients were CTC positive before initial surgery, who had ascites or had residual tumor masses after surgery (Table 3). Six months after completion of their adjuvant chemotherapy, significantly more FIGO IV than FIGO II or III patients and significantly more patients who did not respond to the treatment were CTC positive (Table 4). The presence of CTC before initial surgery had no influence on patients' survival. Patients still presenting CTC six months after chemotherapy had a significantly shorter overall and progression free survival (patients were followed for a median of 31 months).

Results obtained with RT-qPCR and immunocytochemistry perfectly agreed in 35/57 (61%) blood samples (53% both negative, 9% both positive). 15/57 (26%) samples were identified by ICC as CTC-positive, but not with RT-qPCR. 7/57 (12%) samples were only identified by RT-qPCR. Interestingly, patients with CTC identified by RT-qPCR only had a significantly worse prognosis than patients with both RT-qPCR- and ICC-negative results.

Cyclophilin C (PPIC) was predominantly over-expressed in the blood of CTC+ ovarian cancer patients. These patients had a significantly shorter overall and progression free survival compared to those CTC+ patients without over-expression. In contrast, p-Cadherin (CDH3) gene expression had a reverse impact on patient outcome. PPIC and CDH3 gene expression might characterize CTC with different metastatic potential.

The multiple survival analysis using a proportional hazards Cox regression model including age, FIGO stage, residual tumor, grade, and marker gene over-expression before and after treatment revealed that stage FIGO IV, peritonealcarcinomatosis, and the presence of circulating tumor cells as identified by RT-qPCR after chemotherapy are independent predictors of reduced progression free survival. Patient age, peritonealcarcinomatosis, and circulating tumor cells still present after chemotherapy are independent predictors of reduced overall survival (Table 5).

In summary, starting from a whole genome expression analysis of ovarian cancer tissue samples and peripheral blood mononuclear cells we identified 11 novel gene marker for the detection and characterization of circulating tumor cells in the blood of ovarian cancer patients. Using this gene panel for multimarker RT-qPCR analysis, CTC were detected in blood samples taken before initial treatment in 24% of the patients. The presence of CTC six months after completion of chemotherapy reflected worse patient outcome. The molecular detection of CTC provided additional information where standard immunocytochemistry failed. The over-expression of two genes (PPIC and CDH3) has opposing impact on patient outcome. Therefore, PPIC and CDH3 gene expression might characterize CTC with different metastatic potential.

TABLE 1
CTC before initial surgery and six months after chemotherapy

| | Number of Patients (%) | | |
|---|---|---|---|
| number of overexpressed | | Before initial | 6 months after |
| gene markers | | surgery | chemo |

| | | | | |
|---|---|---|---|---|
| ≧1 | 56 | (24.3) | 21 | (18.3) |
| 1 | 42 | (18.3) | 18 | (15.7) |
| 2 | 8 | (3.5) | 3 | (1.2) |
| 3 | 2 | (0.8) | | |
| 4 | 1 | (0.4) | | |
| 5 | 1 | (0.4) | | |
| 6 | 2 | (0.8) | | |

TABLE 2
Overexpression of 11 gene markers and of EpCAM before initial
surgery and six months after chemotherapy

| | | Before initial | | | 6 months after | | |
|---|---|---|---|---|---|---|---|
| | | surgery | | | chemo | | |
| Gene | | | | % | | | % |
| symbol | Gene name | N | % | pos | N | % | pos |
| PPIC | Cyclophilin C | 39 | 17.0 | 69.6 | 15 | 13.0 | 71.4 |
| GPX8 | Probable glutathione | 14 | 6.1 | 25.0 | 1 | 0.9 | 4.8 |
| | peroxidase 8 | | | | | | |
| CDH3 | Cadherin-3 | 9 | 3.9 | 16.1 | 2 | 1.3 | 9.5 |
| TUSC3 | Tumor suppressor | 7 | 3.0 | 12.5 | 1 | 0.9 | 4.8 |
| | candidate 3 | | | | | | |
| AGR2 | Anterior gradient | 1 | 0.4 | 0.2 | 1 | 0.9 | 4.8 |
| | protein 2 homolog | | | | | | |
| COL3A1 | Collagen alpha- | 3 | 1.3 | 5.4 | 1 | 0.9 | 4.8 |
| | 1(III) chain | | | | | | |
| LAMB1 | Laminin subunit | 3 | 1.3 | 5.4 | 1 | 0.9 | 4.8 |
| | beta-1 | | | | | | |
| MAM | Mammaglobin A | 2 | 0.9 | 3.6 | 0 | | |
| TFF1 | Trefoil factor 1 | 1 | 0.4 | 0.2 | 0 | | |
| BAIAP2L1 | Brain-specific | 0 | | | 0 | | |
| | angiogenesis | | | | | | |
| | inhibitor | | | | | | |
| | 1-associated | | | | | | |
| | protein 2-like | | | | | | |
| | protein 1 | | | | | | |
| ESRP2 | Epithelial splicing | 2 | 0.9 | 3.6 | 1 | 0.9 | 4.8 |
| | regulatory protein 2 | | | | | | |
| EPCAM | Epithelial cell | 4 | 1.7 | 7.1 | 1 | 0.9 | 4.8 |
| | adhesion molecule | | | | | | |

TABLE 3
Correlation of CTC presence before initial
surgery with clinical parameters

| | Number of patients (%) | | | | |
|---|---|---|---|---|---|
| | CTC positive | | CTC negative | | p-value |
| Total | 56 | (24) | 174 | (76) | |
| Mean age (yrs) | 59.2 | (±12.7) | 58.4 | (±11.5) | n.s. |
| FIGO stage | | | | | |
| II/III (N = 191) | 45 | (24) | 146 | (76) | n.s |
| IV (N = 39) | 11 | (28) | 28 | (72) | |
| Therapy response | | | | | |
| yes (N = 172) | 41 | (24) | 131 | (76) | n.s |
| no (N = 57) | 15 | (26) | 42 | (74) | |
| Subsequent recurrence | | | | | |
| yes (N = 148) | 39 | (26) | 109 | (74) | n.s |
| no (N = 82) | 17 | (21) | 65 | (79) | |
| Residual disease | | | | | |
| yes (N = 73) | 24 | (33) | 49 | (67) | 0.032 |
| no (N = 156) | 31 | (20) | 125 | (80) | |
| Peritonealcarcinomatosis | | | | | |
| yes (N = 155) | 41 | (27) | 114 | (73) | n.s. |
| no (N = 74) | 14 | (19) | 60 | (81) | |
| Ascites | | | | | |
| yes (N = 173) | 49 | (28) | 124 | (72) | 0.014 |
| no (N = 57) | 7 | (12) | 50 | (88) | |

TABLE 4
Correlation of CTC presence 6 months after
chemotherapy with clinical parameters

| | Number of patients (%) | | | | |
|---|---|---|---|---|---|
| | CTC positive | | CTC negative | | p-value |
| Total | 21 | (18) | 94 | (82) | |
| Mean age (yrs) | 63.8 | (±12.2) | 56.1 | (±11.3) | 0.007 |
| FIGO stage | | | | | |
| II/III (N = 99) | 14 | (14) | 85 | (86) | 0.01 |
| IV (N = 16) | 7 | (44) | 9 | (66) | |
| Therapy response | | | | | |
| yes (N = 95) | 13 | (14) | 82 | (86) | 0.01 |
| no (N = 20) | 8 | (40) | 12 | (60) | |
| Subsequent recurrence | | | | | |
| yes (N = 76) | 17 | (22) | 59 | (78) | n.s. |
| no (N = 39) | 4 | (10) | 35 | (90) | |
| Residual disease | | | | | |
| yes (N = 84) | 15 | (18) | 69 | (82) | n.s |
| no (N = 30) | 6 | (20) | 24 | (80) | |
| Peritonealcarcinomatosis | | | | | |
| yes (N = 78) | 18 | (23) | 60 | (77) | n.s |
| no (N = 36) | 3 | (8) | 33 | (92) | |
| Ascites | | | | | |
| yes (N = 92) | 16 | (17) | 76 | (83) | n.s |
| no (N = 23) | 5 | (22) | 18 | (78) | |

TABLE 5
Cox's proportional hazard regression models for progression free and overall survival

| | Univariate Cox-Regression | | | Multiple Cox-Regression | | |
|---|---|---|---|---|---|---|
| | HR | CI 95% | p | HR | CI 95% | p |
| Age | 1.012 | 0.998-1.026 | 0.102 | | | |
| FIGO IV vs. II/III | 2.269 | 1.550-3.323 | <0.001 | 2.725 | 1.436-5.168 | 0.002 |
| Residual tumor yes vs. | 2.117 | 1.527-2.934 | <0.001 | | | |
| no | | | | | | |
| Grade 3 vs. 1/2 | 1.525 | 1.058-2.198 | 0.024 | | | |
| Peritonealcarcinomatosis | 3.112 | 2.105-4.600 | <0.001 | 3.550 | 1.946-6.478 | <0.001 |
| yes vs. no | | | | | | |
| Ascites yes vs.no | 1.443 | 0.975-2.135 | 0.067 | | | |
| CTC before surgery | 1.325 | 0.918-1.912 | 0.133 | | | |
| CTC after chemo | 2.268 | 1.312-3.922 | 0.003 | 2.063 | 1.175-3.619 | 0.012 |

Methods

Selection of Candidate Genes

Microarray data was obtained from ovarian cancer tissues (35 patients) and from peripheral blood mononuclear cells (20 healthy female donors, 35 patients). 25 markers with low/absent gene expression in healthy PBMCs and high expression in ovarian cancer tissues were selected as candidate gene markers for the detection of circulating tumor cells in the blood of ovarian cancer patients. 15 additional markers were selected from results generated in a previous national project. The expression of these 40 differentially expressed genes was validated in PBMCs obtained from 20 patients with benign ovarian diseases with RT-qPCR. 11 gene markers that had no expression in the benign PBMC remained for further analysis. EpCAM (Epithelial cell adhesion molecule) was analysed as additional gene marker to compare results obtained from RT-qPCR and from immunocytochemistry.

Blood Sample Preparation

25 ml peripheral blood was drawn in Vacuette EDTA tubes and processed within 30 minutes. The monocyte blood fraction containing epithelial cells was enriched using a density gradient centrifugation as described [1]. After two washings with each 50 ml PBS, the cell pellet was lysed in 350 μl RLT buffer (Qiagen, Hilden, Germany). Automated purification of RNA using the RNeasy Mini Kit was performed by the Qiacube system (Qiagen, Hilden, Germany). Quality of the RNA samples was assessed on the 2100 Bioanalyzer using the RNA 6000 Nano LabChip Kit (Agilent Technologies, Waldbronn, Germany). Template cDNA was generated with M-MLV Reverse Transcriptase, RNase H Minus (Promega, Madison Wis., USA) and random nonamers (Sigma-Aldrich, Steinheim, Germany) as primers.

RT-qPCR

RT-qPCR was performed on the 7900HT Fast Real-Time PCR System in duplicate reactions using TaqMan® Pre-Developed Assay Reagents (see Table 6) and TaqMan® Universal PCR Master Mix, No AmpErase® UNG (all purchased from Applied Biosystems, Foster City Calif., USA) as recommended by the manufacturer. Raw data were analyzed with the AB 7900 Sequence Detection Software version 2.2.2 using automatic baseline correction and cycle threshold (Ct) setting. Gene expression was normalized to the geometric mean of the endogenous control gene expression levels (GAPDH, ACTB, and TBP). Low-level gene expression in the healthy control samples required the introduction of a cut-off threshold value to separate the cancer patient group from the healthy control group:

As proposed by Mikhitarian et al. [2], a threshold value TX for each gene X was set to three standard deviations from the mean dCtX value in the control group. A tumor patient was considered positive for the molecular analysis of gene X if dCtX was below the defined threshold value TX.

TABLE 6
TaqMan Assay IDs

| | |
|---|---|
| AGR2 | Hs00180702_m1 |
| BAIAP2L1 | Hs00218959_m1 |
| CDH3 | Hs00354998_m1 |
| COL3A1 | Hs00164103_m1 |
| ESRP2 | Hs00227840_m1 |
| GPX8 | Hs00380670_m1 |
| LAMB1 | Hs00158620_m1 |
| PPIC | Hs00181460_m1 |
| SCGB2A2 | Hs00267190_m1 |
| TACSTD1 | Hs00158980_m1 |
| TFF1 | Hs00170216_m1 |
| TUSC3 | Hs00954406_m1 |
| EpCAM | Hs00158980_m1 |

Although the present invention has been described in considerable detail with reference to certain preferred embodiments, other embodiments are possible. Therefore, the scope of the appended claims should not be limited to the description of preferred embodiments contained in this disclosure. All references cited herein are incorporated by reference in their entirety.

### REFERENCES

1. Brandt B, Griwatz C: Two-layer buoyant density centrifugation gradient for enrichment of prostate-derived cells and cell clusters from peripheral blood. Clin Chem 1996, 42(11):1881-1882.

2. Mikhitarian K, Martin R H, Ruppel M B, Gillanders W E, Hoda R, Schutte del H, Callahan K, Mitas M, Cole D J: Detection of mammaglobin mRNA in peripheral blood is associated with high grade breast cancer: interim results of a prospective cohort study. BMC Cancer 2008, 8:55.

3. Lee J, Kim S S: Current implications of cyclophilins in human cancers. J Exp Clin Cancer Res, 29:97.

4. Mi Z, Oliver T, Guo H, Gao C, Kuo P C: Thrombin-cleaved COOH(—) terminal osteopontin peptide binds with cyclophilin C to CD147 in murine breast cancer cells. Cancer Res 2007, 67(9):4088-4097.

5. Aberger F, Weidinger G, Grunz H, Richter K: Anterior specification of embryonic ectoderm: the role of the Xenopus cement gland-specific gene XAG-2. Mech Dev 1998, 72(1-2):115-130.

6. Smirnov D A, Foulk B W, Doyle G V, Connelly M C, Terstappen L W, O'Hara S M: Global gene expression profiling of circulating endothelial cells in patients with metastatic carcinomas. Cancer Res 2006, 66(6):2918-2922.

7. Watson M A, Fleming T P: Mammaglobin, a mammary-specific member of the uteroglobin gene family, is overexpressed in human breast cancer. Cancer Res 1996, 56(4):860-865.

8. Klug J, Beier H M, Bernard A, Chilton B S, Fleming T P, Lehrer R I, Miele L, Pattabiraman N, Singh G: Uteroglobin/Clara cell 10-kDa family of proteins: nomenclature committee report. Ann N Y Acad Sci 2000, 923:348-354.

9. Brown N M, Stenzel T T, Friedman P N, Henslee J, Huper G, Marks J R: Evaluation of expression based markers for the detection of breast cancer cells. Breast Cancer Res Treat 2006, 97(1):41-47.

10. Grunewald K, Haun M, Urbanek M, Fiegl M, Muller-Holzner E, Gunsilius E, Dunser M, Marth C, Gastl G: Mammaglobin gene expression: a superior marker of breast cancer cells in peripheral blood in comparison to epidermal-growth-factor receptor and cytokeratin-19. Lab Invest 2000, 80(7):1071-1077.

11. Zafrakas M, Petschke B, Donner A, Fritzsche F, Kristiansen G, Knuchel R, Dahl E: Expression analysis of mammaglobin A (SCGB2A2) and lipophilin B (SCGB1 D2) in more than 300 human tumors and matching normal tissues reveals their co-expression in gynecologic malignancies. BMC Cancer 2006, 6:88.

12. Abdou A G, Aiad H A, Sultan S M: pS2 (TFF1) expression in prostate carcinoma: correlation with steroid receptor status. Apmis 2008, 116(11):961-971.

13. Warzecha C C, Sato T K, Nabet B, Hogenesch J B, Carstens R P: ESRP1 and ESRP2 are epithelial cell-type-specific regulators of FGFR2 splicing. Mol Cell 2009, 33(5):591-601.

1. A method of determining gynaecologic tumor disease in a subject, comprising:
providing a sample of peripheral blood of the subject,
measuring PPIC expression of cells in the sample, and
comparing the PPIC expression to a reference value, the PPIC overexpression being indicative of a gynecologic tumor disease and/or disease progression.

2. The method of claim 1, wherein the sample is obtained from a blood fraction containing mononuclear cells.

3. The method of claim 2, wherein the blood fraction is a PBMC fraction.

4. The method of claim 1, further comprising the step of determining CDH3 expression of said CTC, wherein the CDH3 expression is indicative of an improved prognosis.

5. The method of claim 1, further comprising the step of identifying at least one further marker in the sample, wherein the marker is selected from the group consisting of GPX8, TUSC3, AGR2, COL3A1, LAMB1, MAM, TFF1, BAIAP2L1, ESRP2 and EpCAM.

6. The method of claim 1, wherein the PPIC expression is quantitatively determined.

7. The method of claim 1, wherein nucleic acid and/or protein expression is determined.

8. The method of claim 7, wherein nucleic acid and/or protein expression is determined using a method selected from the group consisting of nucleic acid amplification, RT-qPCR, microarray testing, an immunoassay, protein array testing, an immunocytochemistry method, and an immunohistochemistry method.

9. The method of claim 1, wherein the sample contains at least one tumor cell per milliliter.

10. The method of claim 1, wherein the reference value is determined in a sample of a healthy subject.

11. The method of claim 1, wherein the subject is determined to be at risk of ovarian, breast, endometrial or cervical tumor disease.

12. The method of claim 1, wherein the subject is suffering from early stage cancer.

13. The method of claim 1, wherein the subject is undergoing or has received chemotherapy, and the risk of disease progression is determined.

14. The method of claim 1, wherein the subject is suffering from minimal residual disease.

15. The method of claim 1, wherein the PPIC overexpression is indicative of an increased metastatic potential associated with a shortened survival time.

16. The method of claim 1, wherein the metastatic potential is determined in a gynecologic cancer patient at risk of disease progression.
